(19) United States
(12) Patent Application Publication
(10) Pub. No.: US 2015/0237890 A1
(43) Pub. Date: Aug. 27, 2015

(54) SYMBIOTIC SHRIMP AND ALGAE GROWTH SYSTEM

(71) Applicant: Once Innovations, Inc., Plymouth, MN (US)

(72) Inventor: Zdenko Grajcar, Orono, MN (US)

(21) Appl. No.: 14/425,332

(22) PCT Filed: Sep. 6, 2013
(86) PCT No.: PCT/US2013/058511

§ 371 Date: Mar. 3, 2015

Related U.S. Application Data
(60) Provisional application No. 61/698,029, filed on Sep. 7, 2012.
(60) Provisional application No. 61/698,074, filed on Sep. 7, 2012.

(51) Int. Cl. A23K 1/18; F21K 99/00; A01H 13/00; A01K 61/00; A01K 61/02

(52) U.S. Cl. CPC A23K 1/188; A01K 61/00; A01K 61/005; A01K 61/02; A01H 13/00; F21K 9/00

(57) ABSTRACT

A system for enhancing the growth of aquatic life that includes first and second raceways that both extend from an inlet to an outlet with a channel therebetween and hold water. The raceways are in side by side relation and are in fluid communication with one another. The first raceway has a living food source within the water and the second raceway has aquatic life within the water. Lighting assemblies are provided in each raceway to enhance both the living food source and the aquatic life by using predetermined wavelengths of light.

### CROSS-REFERENCE TO RELATED APPLICATIONS

This application claims the benefit of U.S. Provisional Patent Application 61/698,029 entitled “Symbiotic Shrimp and Algae Growth System” filed on Sep. 7, 2012, and the benefit of U.S. Provisional Patent Application 61/698,074 entitled “Aquatic System for Manipulating Psychological and Physiological Effects in Aquatic Life”, also filed on Sep. 7, 2012, the entire contents of each of which are incorporated herein by reference in their entirety.

### BACKGROUND

This invention relates to aquaculture. More specifically this invention relates to a symbiotic shrimp and algae growth system.

Seafood has always been a source of food for humans. Further seafood has been proven to be a healthy source of food and as a result seafood consumption is increasing worldwide. Still, as the environment and in particular waterways become more polluted over time, raising and harvesting seafood to meet the rising demands of consumers becomes more and more difficult. As a result of this need aquaculture or aquatic farming of seafood has grown in popularity.

Typically an aquatic farm will consist of a plurality of side by side raceways containing aquatic life, including but not limited to shrimp, tilapia, salmon, trout, other fresh water and salt water fish or the like. These raceways are typically located outdoors and water is constantly run through the raceway for filtration purposes. The aquatic life then eats a food source, such as algae provided and can be harvested once fully grown.

In aquaculture systems for growing aquatic life, water is constantly conveyed through raceways where fish, shrimp or other aquatic life is grown and harvested. Typical raceways are rectangular in shape and located in parallel with one another. Water is constantly run from an inlet side of the raceway to an outlet end.

Problems exist in these systems because water must be constantly run through the raceways to keep the water quality at a high level. In particular, fish and/or shrimp waste accumulates in the system and must be continually cleaned.

Further, these aquaculture systems must be outdoors in order to provide the sunshine and light needed in order to grow food for the aquatic life within the raceways. This is often problematic because on rainy and cloudy days sunlight can be scarce and thus optimum growing conditions for both the aquatic life and their food source is not achieved. Further, water temperature is difficult to moderate in an outdoor environment also, with the water temperature constantly attempting to reach equilibrium with the outside temperature. This again does not provide optimum growing conditions for the aquatic life or food within the raceways. In addition, when the sun is providing sunlight the light hits the top surface of the water that has reflective properties, again providing growing conditions that are difficult to control and not optimum for growing.

Another problem exists in providing a food source for the aquatic life. In particular, for aquatic life such as shrimp, their food source is algae that absorbs sunlight in order to grow. The growth rate of the algae has a direct effect on the amount of shrimp that can survive in a raceway. If the algae does not grow at a sufficient rate, the shrimp do not have enough food and either additional food/algae must be placed into the raceway or fewer shrimp are able to survive.

Known in the art is that plants such as algae absorb different frequencies of light to cause photosynthesis to occur. In particular photsynthetically active radiation (PAR) is radiation in the spectral range from approximately 400 nanometers (nm) to 700 nm. Also known in the art is that chlorophyll, the most abundant plant pigment and the pigment responsible for plant metabolism is most efficient at capturing red and blue light. Therefore algae growth is optimized when bombarded with red and blue wavelength radiation.

Also known in the art is that animals similarly react to different wavelengths of radiation for growth. For example, white light can stimulate activity and breeding in animals. Similar to plants, red and blue lights can be shown to enhance growth characteristics in animals.

Thus, a need in the art exists for an aquaculture system that is easy to filter and clean and allows for indoor growing of aquatic life. In particular a need exists to provide a controlled environment for growing aquatic life to maximize aquatic life yield, size and taste in an efficient and self-sustaining manner.

Thus, a principle object of the present invention is to provide a lighting assembly that provides varied wavelengths of light to optimize aquatic life growth and yield.

Another object of the present invention is to provide an inexpensive lighting assembly for optimizing physiological and psychological effects on aquatic life.

Another object of the present invention is to provide optimum lighting for aquatic life within an aquaculture system.

These and other objects, features, and advantages will become apparent from the specification and claims.

### SUMMARY OF THE INVENTION

An aquatic system having a first raceway containing an aquatic life food source disposed therein. The system additionally has a second raceway having aquatic life disposed therein in fluid communication with the first raceway. Lighting assemblies having sets of LED lights are disposed within both the first and second raceways to provide lighting to the food source and aquatic life disposed therein. The lighting assemblies are tuned to wavelengths of light associated with the food source and aquatic life respectfully to maximize the food production and optimize the growth, health and taste of the aquatic life.

### BRIEF DESCRIPTION OF THE DRAWINGS

FIG. 1 is a side plan view of an aquatic system;

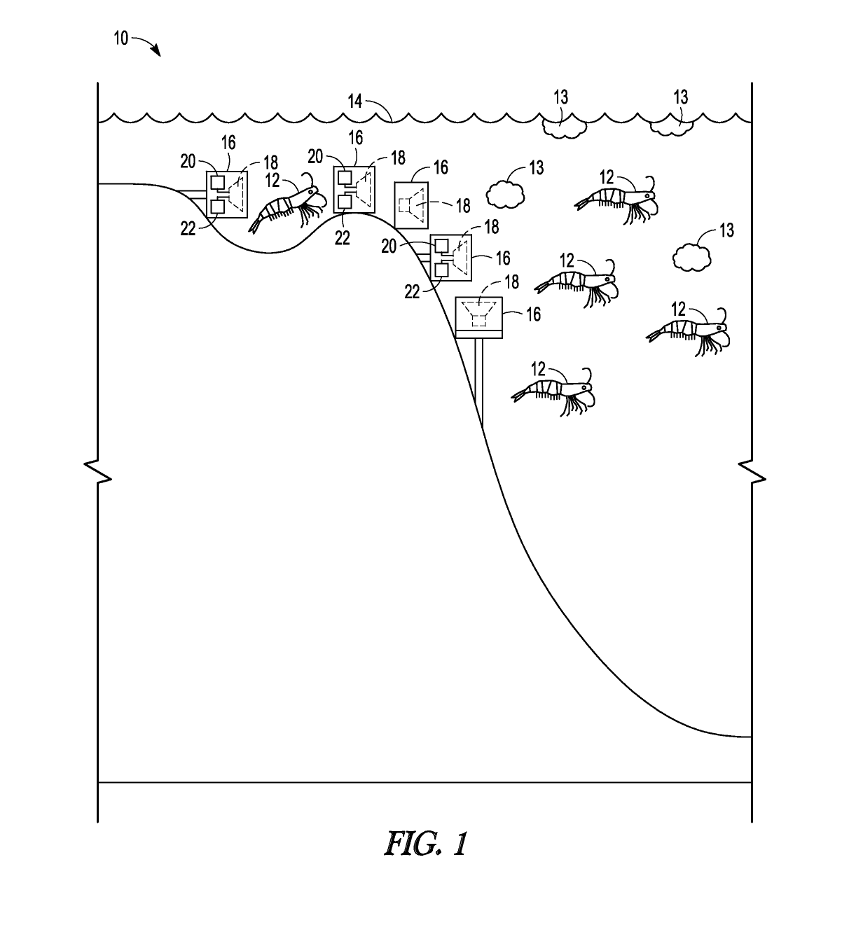

FIG. 2 is a side plan view of an aquatic system;

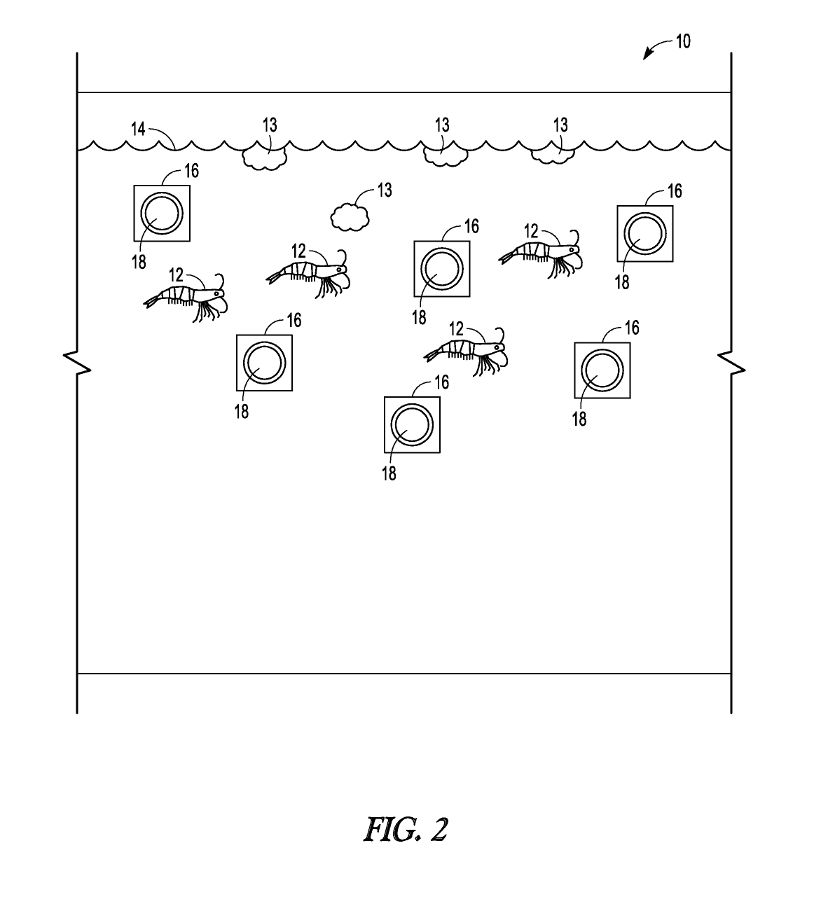

FIG. 3 is a side plan view of an aquatic system;

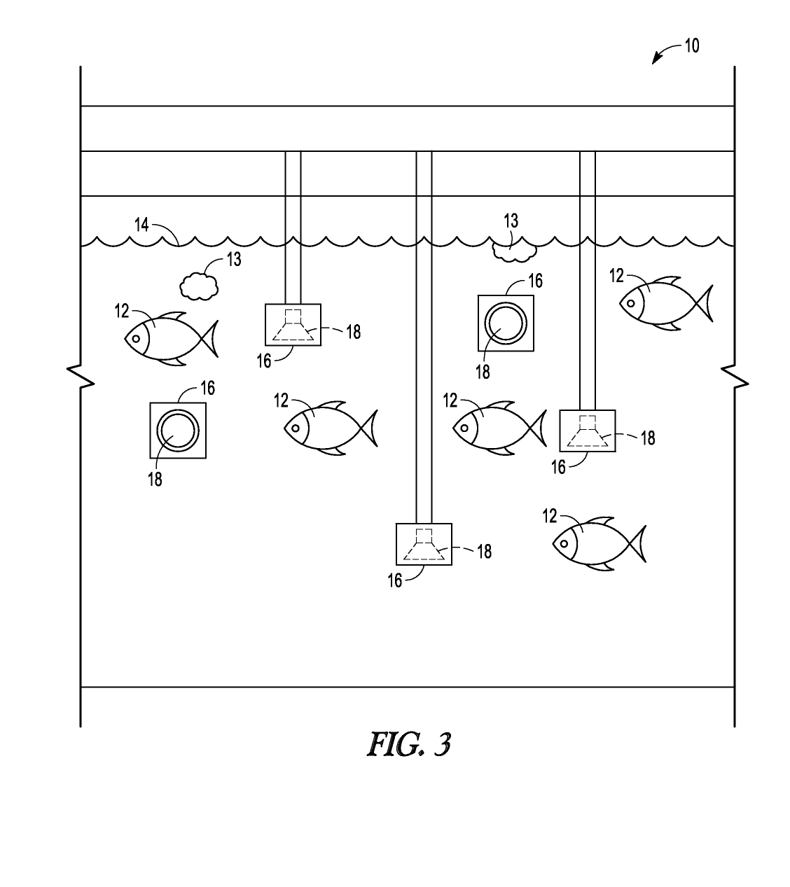

FIG. 4 is a top plan view of a raceway aquatic system; and

FIG. 5 is a side plan view of a raceway aquatic system.

### DETAILED DESCRIPTION OF A PREFERRED EMBODIMENT OF THE INVENTION

The figures show an aquatic system 10 that contains a plurality of aquatic life 12 and a food source 13 in water 14. The aquatic life 12 may be any aquatic life, including, but not limited to shrimp, salmon, trout, tilapia, any other salt or fresh water fish or the like. The food source 13 includes, but is not limited to algae and other plant and/or animal life consumed by aquatic life. The water 14 may be fresh or salt water. Similarly, the water 14 may be in a body of water such as an ocean, bay, sea, gulf, lake, river, stream or the like, or alternatively be within a manmade structure such as a raceway, indoor raceway, indoor pool, or the like.

At least one lighting assembly 16 is placed in, on, by or adjacent to the water 14 such that lights 18 within the lighting assembly 16 reach and/or are absorbed by the aquatic life 12. In particular, lighting assemblies can be placed in the bed of a body of water, mounted on mounting equipment, hung on a dock, placed in an encasement in a raceway or the like to be placed in water adjacent to a food source 13 and/or aquatic life 12 such that the food source 13 and/or aquatic life 12 receives light emitted by the lighting assemblies 16.

Preferably the lights 18 are light emitting diodes (LEDs) that are able to be controlled to produce any desired wavelength of visible light having a wavelength between 400-700 nm. In particular the lighting assemblies 16 are built with an AC driven power source and as presented in U.S. Pat. Publ. 2011/0273098 to Grajcar; U.S. Pat. Publ. 2011/0210678 to Grajcar; U.S. Pat. Publ. No. 2011/0109244 to Grajcar; USSN 13/452332 to Grajcar; and/or USSN 61/570,552 to Grajcar, each of which is fully incorporated herein.

As a result of the technology presented in the listed patent applications, each lighting assembly 16 has a plurality of LED lights 18 that are powered by an AC driver 20 having a dimmer 22 such that the wavelengths of the lights 18 can be controlled to provide 400-700 nm wavelength light as desired and at predetermined intervals as desired using an AC power source. Preferably light at a wavelength of 490 nm is provided.

In operation, a particular aquatic life 12, such as shrimp, a salt water fish, a fresh water fish or the like, or food source 13 such as algae is analyzed to determine predetermined wavelengths of light and/or combination of wavelengths of light to effect psychological and physiological functioning of the aquatic life 12 or food source 13. For algae and to attract smaller fish and marine animals this wavelength is shown to be 490 nm. Based on this analysis a predetermined coloring pattern is determined to be emitted by the lighting assemblies 16. The lighting assemblies 16 are then actuated to emit the predetermined coloring pattern at predetermined intervals. The aquatic life 12 and food source 13 then receives this light in order to alter the psychological and physiological functioning of the aquatic life 12 to maximize or enhance growth, color or yield of the aquatic life 12.

The predetermined color pattern can include but is not limited to having red wavelength light, blue wavelength light, or green-blue light having a wavelength of 490 nm. Further the predetermined color pattern can present the use of only one wavelength or color of light.

The predetermined color pattern in one embodiment is based on maximizing the growth of the aquatic life 12. In yet another embodiment the predetermined color pattern is based on maximizing the yield of aquatic life 12. This yield includes increases in yield as a result of direct lighting from lighting assemblies 16 on the aquatic life 12, while other lighting assemblies 16 provide a predetermined color pattern to enhance and to optimize the growing of a food source 13 such as algae in the water 14 to maximize the food consumed by the aquatic life 12 thus increasing yield.

In yet another embodiment where the aquatic life 12 is shrimp the predetermined color pattern is based on enhancing the shrimp color to be pink. In yet another embodiment the predetermined color pattern increases the fatty acids in an aquatic life 12 such as shrimp. In another embodiment the predetermined color pattern decreases enzyme activity in an aquatic life such as shrimp to minimize disease such as melanin to prevent black spots. In other embodiments the predetermined color pattern controls the rate of molting in a shrimp in order to yield more edible shrimp meat per pound of whole shrimp.

Thus provided is an aquatic system 10 that utilizes AC driven lighting assemblies 16 to power LED lighting to provide predetermined wavelengths/color patterns of light to assist in raising and harvesting aquatic life 12 such as shrimp. The lighting assemblies 16 are used to maximize growth, enhance color, minimize disease and/or increase yield of the aquatic life 12. Similarly color patterns can be used to increase the growth of a food source 13, which in turn acts to enhance growth and increase yield of aquatic life 12. In addition, by using AC driven lighting assemblies 16 as presented in the applications incorporated into this application, costs are minimized as compared to DC driven lighting assemblies that are able to provide varied wavelength outputs. Thus, at the very least, all of the stated objects have been met.

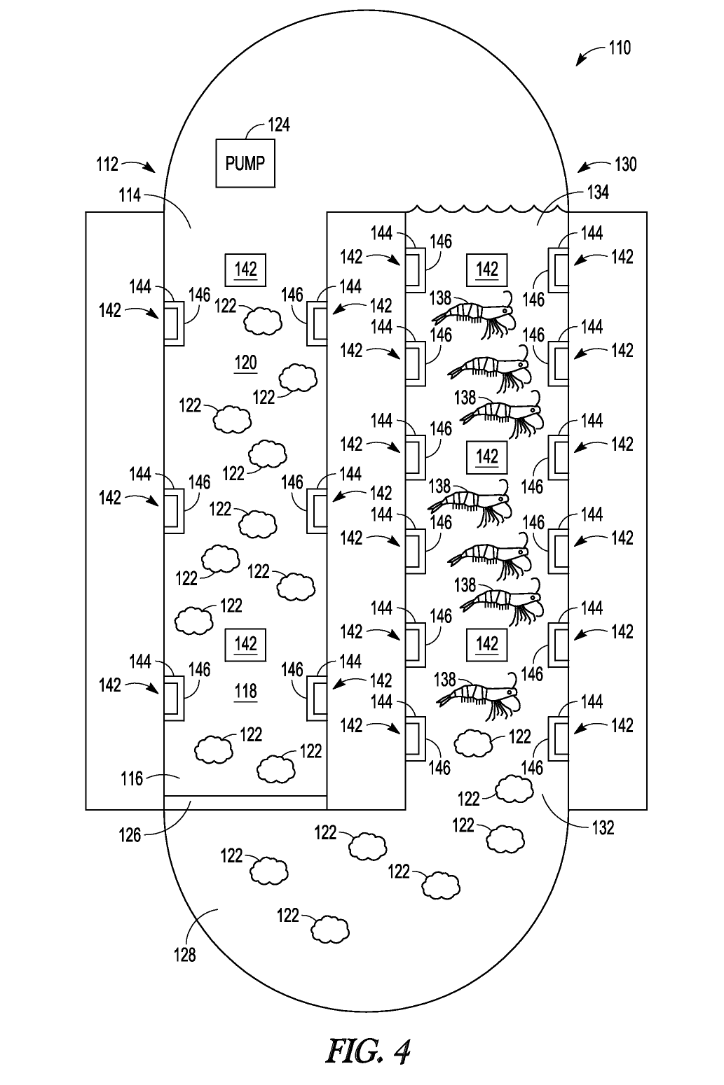

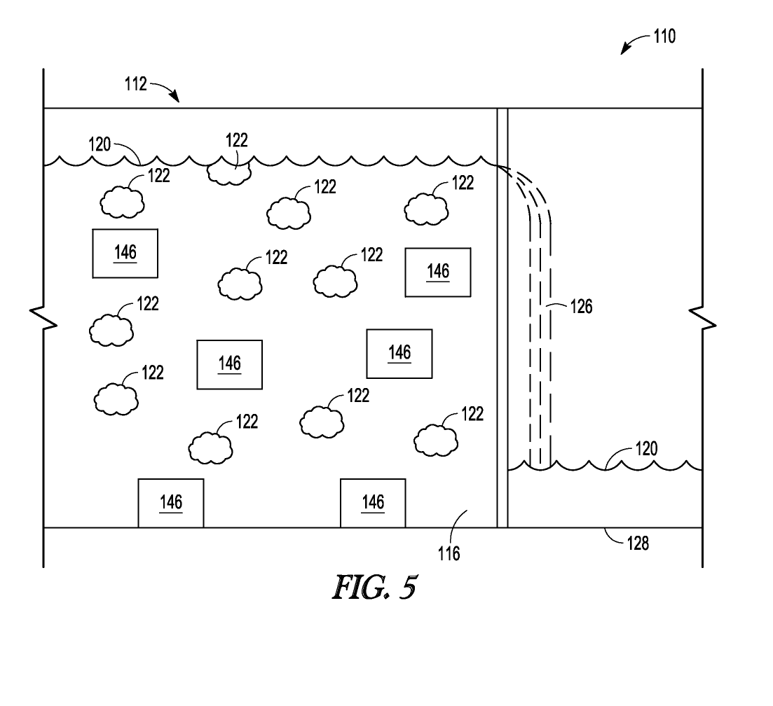

In an alternative embodiment as shown in FIGS. 4 and 5 show an aquatic system 110 having a first raceway 112. The first raceway 112 extends from an inlet 114 to an outlet 116 with a channel 118 for holding water 120 disposed therebetween. The first raceway 112 contains or has a food source 122 disposed therein. In a preferred embodiment the food source 122 is algae. A pump 124 pumps water 120 containing the food source 122 over an aerating waterfall 126 adjacent the first raceway to a first flow path 128 that conveys the water to a second raceway 130. While only two raceways are shown in the Figure, it is well known in the art that multiple raceways are utilized in association with aquatic systems 110.

The second raceway 130 also extends from an inlet 132 to an outlet 134 with a channel 136 for holding water 120 disposed therebetween. The second raceway contains aquatic life 138 to be harvested for consumption by the general public. Aquatic life 138 includes, but is not limited to, shrimp, trout, fresh water fish, salt water fish and the like. This aquatic life 138 feeds off of the food source 122 conveyed to the second raceway 30 and lives within the channel 136. The aquatic life 138 additionally produces waste and high mineral molt into the water 120 which contains nitrogen and nitrates that are pumped to a second fluid path 140 to the inlet 114 of the first raceway 112. This nitrogen and nitrate filled water 120 provides an excellent medium for growing the food source 122 and in particular algae.

A plurality of lighting assemblies 142 that each have a plurality of light emitting diodes (LEDs) are disposed throughout channels 118 and 136 of the first and second raceways 112 and 130 to provide lighting for both the food source 122 and the aquatic life 138. The assemblies 142 are suspended in the water 120 at various depths and/or are mounted on the outside of enclosures 144 having a sidewall 146 made of transparent material such as a Plexiglas® window through which the light from the LED lighting assembly 142 shines.

The assemblies 142 are constructed in or attached to or provided in the raceways 112 and 130 in any manner These include methods that are presented in U.S. provisional application Ser. No. 61/570,552 to Grajcar that is incorporated herein. Similarly, the assemblies 142 are comprised in any manner and in one embodiment are sealed as best shown in U.S. patent application Ser. No. 13/011,927 to Grajcar that is incorporated herein. The assemblies in multiple embodiments are also constructed consistent with USSN 12/785,498 to Grajcar, 61/233,829 to Grajcar and 61/234,094 to Grajcar all of which are incorporated herein.

The assemblies also can be tuned to accommodate different light wavelength needs of the food source 122 and aquatic life 138 in order to not only stimulate the growth of the food source 122 and aquatic 138, but also to maximize such growth. Such tuning can be accomplished as taught in U.S. patent application Ser. No. 12/824,215 to Grajcar and/or U.S. patent application Ser. No. 12/914,575 to Grajcar, both that are incorporated herein.

In operation the LED light assemblies 142 function to provide lighting within both the first and second raceways 112 and 130. In the first raceway 112 the lighting assemblies 142 operate to provide predetermined amounts of red and blue lighting that optimize algae growth. Specifically the optimum radiation absorption wavelengths are predetermined and the light assemblies 142 provide such wavelengths at predetermined intervals to maximize the growth of the algae. This is accomplished through methods and circuits as described above to control the radiation provided.

Similarly, in the second raceway 130 the light assemblies 142 function to provide lighting at predetermined wavelengths at predetermined intervals to maximize the yield and growth of the aquatic life 138. In this manner the lighting assemblies maximize the food source 122 in the first raceway 112 that is then conveyed to the second raceway 130 to be consumed by the aquatic life 138. The aquatic life 138 then consumes the food source 122 to maximize yield and as a result of the light assemblies 142 maximize growth of the aquatic life 138. Simultaneously the aquatic life 138 produces waste and high mineral molt rich in nitrogen and nitrates. This waste is conveyed to the first raceway where the food source 122 such as algae that can feed off the waste to provide additional food source 122 growth. Therefore, by having the food source 122 consume waste product and by constantly filtering the water, superior light penetration through the water occurs to enhance and optimize the growth of the food source 122 and aquatic life 138.

Thus, fresh or salt water, depending on the aquatic life 138, is circulated in a loop through first and second raceways 112 and 130. The water 120 is pumped with pump 124 continuously from the first or food source raceway 112 over the aerating waterfall 126 to the second or aquatic life raceway 130. Nitrogen and nitrates produced by the aquatic life 138 such as shrimp is then conveyed or pumped back to the first raceway 112 to provide a medium for growing the food source 122 such as algae. The lighting assemblies 142 are then used to stimulate the growth of both the food source 122 and aquatic life 138. Lighting assemblies 142 are suspended in the water at various depths and/or mounted on the outside of enclosures 144 to shine through sidewall 146 made of transparent material such as Plexiglass® as shown in FIG. 5.

Thus presented as a result of the lighting assemblies 142 is a system 110 that allows for the production of aquatic life 138, such as shrimp, indoors in a bio secure environment unimpeded by rain, cloudy days or uncontrolled temperature. The system 110 additionally allows for continuous growth and production of both a food source 122 such as algae and aquatic life 138 such as shrimp without the need to add extra water. Further the lighting assemblies 142 in the water are mounted on or beside the raceways 112 and 130. The lighting assemblies 142 provide predetermined wavelengths of radiation such as red and blue wavelength radiation that is significantly more efficient in growing both the food source 122 and aquatic life 138 than other artificial light over the top of the water 120. In particular by being placed in the water 120 reflection as a result of the water surface properties is eliminated, creating an even more efficient lighting and growing environment. Plus by filtering the water and tuning the light from the lighting assemblies 142, superior light penetration through the water to enhance the growth of the food source 122 and aquatic life 138 is provided. Thus, at the very least, all of the stated objects have been met.

What is claimed is:

1. A system for enhancing the growth of aquatic life comprising:
a first raceway extending from an inlet to an outlet with a channel therebetween holding water;
a second raceway in fluid communication with the first raceway extending from a second inlet to a second outlet with a second channel therebetween holding water;
said first raceway having a living food source within the water and the second raceway having aquatic life within the water; and
at least one lighting assembly associated with the first raceway providing light at a first predetermined wavelength related to enhancing growth of the living food source.

2. The system of claim 1 further comprising at least one lighting assembly within the second raceway providing light at a second predetermined wavelength related to enhancing growth of the aquatic life.

3. The system of claim 2 wherein the first predetermined wavelength and second predetermined wavelength are substantially the same.

4. The system of claim 2 wherein the first predetermined wavelength and second predetermined wavelength are substantially different.

5. The system of claim 1 wherein the first raceway and second raceway are of one piece construction.

6. The system of claim 1 wherein the first predetermined wavelength is between 400-500 nm.

7. The system of claim 1 wherein the first predetermined wavelength is between 600-700 nm.

8. The system of claim 1 further comprising an aerating waterfall disposed between the first raceway and second race to provide oxygen within the system.

9. The system of claim 1 wherein the at least one lighting assembly is suspended within the water of the first raceway.

10. The system of claim 1 wherein the at least one lighting assembly is mounted on an enclosure adjacent a window within the raceway to provide light through the window.

11. The system of claim 1 wherein the at least one lighting assembly is an LED lighting assembly.

12. The system of claim 1 wherein the living food source is algae.

13. The system of claim 1 wherein the aquatic life is selected from a group consisting of shrimp, salmon, trout and tilapia.

14. A method of artificially enhancing the growth of aquatic life in a raceway using a lighting assembly steps comprising:
providing a first raceway having a living food source within water contained by the raceway;
providing a second raceway in fluid communication with the first raceway;
installing at least one lighting assembly in association with the first raceway to provide artificial light in the water of the first raceway;
artificially enhancing the growth of the living food source with the light; and
conveying the living food source from the first raceway to the second raceway for consumption by the aquatic life.

15. The method of claim 14 steps further comprising:
installing at least one lighting assembly in association with the second raceway to provide artificial light in the water of the second raceway to enhance the growth of the aquatic life within the second raceway.

16. The method of claim 14 wherein the living food source is algae.

17. The method of claim 14 wherein the aquatic life is selected from a group consisting of shrimp, salmon, trout and tilapia.

18. The method of claim 14 wherein the lighting assembly is an LED lighting assembly.

19. The method of claim 15 wherein the artificial light in the water of the second raceway increases the fat content of the aquatic life as a way of enhancing the aquatic life.

20. The method of claim 15 wherein the artificial light in the water of the second raceway reduces disease in the aquatic life as a way of enhancing the aquatic life.
